(19) United States
(12) Patent Application Publication
(10) Pub. No.: US 2018/0022724 A1
(43) Pub. Date: Jan. 25, 2018

(54) KETAL COMPOUNDS AND USES THEREOF

(71) Applicant: GFBiochemicals Limited, Valletta, MT (US)

(72) Inventor: Kevin Jon Bechtold, St. Paul, MN (US)

(21) Appl. No.: 15/545,781

(22) PCT Filed: Jan. 29, 2016
(86) PCT No.: PCT/US16/15519

§ 371 Date: Jul. 24, 2017

Related U.S. Application Data
(60) Provisional application No. 62/109,756, filed on Jan. 30, 2015.

(51) Int. Cl. C07D 319/06; C08K 5/1575

(52) U.S. Cl. CPC C07D 319/06; C08K 5/1575

(57) ABSTRACT

Various esterified alkyl ketal ester or hydroxyalkyl ketal ester products are useful as components of organic polymer compositions. The ketal esters are produced in certain transesterifications between alkyl ketal esters and/or hydroxyalkyl ketal esters and polyols, aminoalcohols, polyamines and/or polycarboxylic acids. The products are excellent plasticizers for a variety of organic polymers, notable poly(vinylchloride) plastisols.

### FIELD OF THE INVENTION

The invention relates generally to the preparation of alkyl ketal ester plasticizers.

### BACKGROUND OF THE INVENTION

Many known chemical products such as surfactants, plasticizers, solvents, and polymers are currently manufactured from non-renewable, expensive, petroleum-derived or natural gas-derived feedstock compounds. High raw material costs and uncertainty of future supplies requires the discovery and development of surfactants, plasticizers, solvents, and polymers that can be made from inexpensive renewable biomass-derived feedstocks and by simple chemical methods. Using renewable resources as feedstocks for chemical processes will reduce the demand on non-renewable fossil fuels currently used in the chemical industry and reduce the overall production of carbon dioxide, the most notable greenhouse gas.

A potential source of materials that are useful as chemical building blocks are cyclic ketals and acetals of oxocarboxylates with polyols. Polyhydric alcohols, or polyols, having 1,2 and 1,3 hydroxy conformations can react with a ketone or aldehyde to form a cyclic ketal or an acetal (Carey, F. A. and Sundberg, R. J., “Advanced Organic Chemistry Part B: Reactions and Synthesis” 2nd ed., 1983, Plenum Press, NY, N.Y., p. 544).

Diols such as 1,2-ethane diol (ethylene glycol) and 1,3 propanediol (propylene glycol) are examples of such polyols. Diols having a 1,2 hydroxyl group configuration form dioxolanes when reacted with ketone or aldehyde moieties, while 1,3 diols form dioxanes.

The use of levulinate compounds and glycerol based compounds is particularly useful as both of these starting materials arise from renewable feedstocks. Further, the ketal reaction products are useful for synthesis of a wide variety of surfactants, plasticizers, polymers, and the like. Other reaction products of oxocarboxylates (such as pyruvic acid, acetoacetic acid, or esters thereof, and the like) with triols (such as trimethylolpropane, trimethylolethane, and the like) are disclosed in International Patent Application No. PCT/US08/75225. The methods employed to synthesize these compounds involve the formation of one mole of water with each mole of ketal formed. Likewise, polyketal compounds are formed from oxocarboxylates and tetrols and higher polyols using similar methods, with one mole of water formed for each mole of ketal functionality formed. Polyketal compounds are described in International Patent Application No. PCT/US08/079337. One example of a polyketal is a bisketal formed from a levulinate ester and erythritol (or a stereoisomer thereof):

Synthetic routes to form ketals of oxocarboxylic acids or the esters thereof are described in International Patent Application No. PCT/US08/079083. The methodology disclosed therein employs very low levels of acid catalyst and certain stoichiometric ratios of oxocarboxylate to polyol to result in high yields of ketal compounds with short reaction times. However, this methodology, as well as previous methods used to form ketals from oxocarboxylates and polyols, necessarily involves the formation of water in conjunction with formation of the ketal end product. Because ketal formation is reversible in the presence of water and the acid catalyst, rigorous removal of water is necessary in order to drive the reaction and maintain high yields and product stability. Additionally, the main side products in the reaction of tetrols and higher polyols to form polyketals are typically those where less than the full desired complement of oxocarboxylate is reacted—e.g., a tetrol such as erythritol or diglycerol having one ketal functionality instead of two; or a hexitol such as mannitol having one or two ketal functionalities instead of three. Such side products are difficult to separate from the desired end product, necessitating fractionation. Further, the free hydroxyl groups present in these side products can undergo side reactions in subsequent polymerization reactions or create incompatibility with one or more formulation components in the application of bisketal and trisketal compounds as plasticizers, solvents, and the like.

Additionally, the structural variation of the ketal and polyketal compounds disclosed in the above cited patent applications and publications are limited to the variation in the polyol and oxocarboxylate compounds employed.

It is desirable to provide new starting materials and synthetic routes to form new varieties of chemical building blocks for monomers, plasticizers, surfactants, and polymers. It is desirable to provide chemical building blocks that arise solely from renewable feedstocks. It is desirable to facilitate synthesis of chemical building blocks that is simple, inexpensive, and scalable for commercialization purposes. It is desirable to avoid the problem of water formation in the ketalization of oxocarboxylic acids or their esters. It is also desirable to obtain such starting materials that are stable and/or have high purity.

### BRIEF SUMMARY OF THE INVENTION

It is desirable to provide commonly used materials, such as surfactants, plasticizers, solvents, and polymers, from renewable feedstocks as a source of chemical building blocks. It is also desirable to provide chemical building blocks that are chemically and thermally stable. Furthermore, chemical building blocks having multiple functionalities for subsequent reactions are often desirable. The ability to provide such materials by simple and reproducible methods that can be carried out with ease is advantageous.

This invention is in one aspect a compound having a structure corresponding to structure I

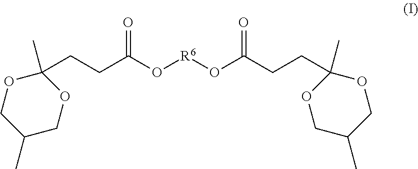

wherein R⁶ is a hydrocarbyl group or a substituted hydrocarbyl group.

The invention in other aspects is a composition comprising a compound of structure I and a polymer.

The invention is also a process for plasticizing a polymer comprising melt or solution blending a polymer and a plasticizing amount of at least one compound of structure I.

In another aspect, the invention is a method of making a compound of structure I comprising:
a. reacting ethyl levulinate with 2-methyl-1,3-propanediol or a ketal or acetal of 2-methyl-1,3-propanediol in the presence of a catalyst;
b. adding a polyol comprising a structure corresponding to R⁶(OH)t to the product of step (a); and
c. effecting a reaction to form a compound having a structure corresponding to structure I, wherein R⁶ is as defined above.

This invention is in one aspect a compound having a structure

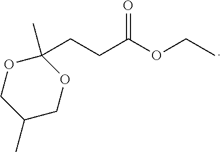

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### DETAILED DESCRIPTION

In the specification and in the claims, the terms “including” and “comprising” are open-ended terms and should be interpreted to mean “including, but not limited to . . . ” These terms encompass the more restrictive terms “consisting essentially of” and “consisting of.”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

This invention is in one aspect a compound having a structure corresponding to structure I

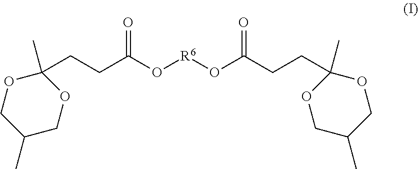

wherein R⁶ is a hydrocarbyl group or a substituted hydrocarbyl group.

In specific embodiments, R⁶ is —(CH₂)—ₘ wherein m is from 2 to 18, especially 2, 3, 4 or 6.

In structure I herein, a “substituted” hydrocarbon or hydrocarbyl group may contain any substituents that do not react with carboxylate groups, hydroxyl groups or amino groups under the conditions of the reactions that form the various products of structure I. Therefore, the substituents should exclude groups such as hydroxyl, primary or secondary amino, mercapto, carboxylic acid or salts or esters thereof, carboxylic acid halides, sulfur- or phosphorus-containing acids, isocyanates and the like. In addition, the substituent groups also should not otherwise interfere with the reactions that form the various products of structure I. Suitable substituents include carbonyl, halogen, tertiary amino, ether, sulfone and the like, among others.

A specific compound according to structure I includes that having the structure

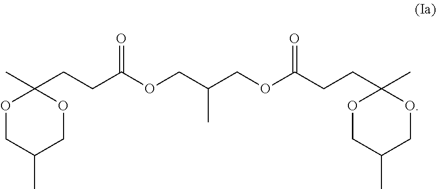 [SMILES: CC(COC(=O)CCC1(C)OCC(C)CO1)COC(=O)CCC1(C)OCC(C)CO1]

Compounds according to structure I can be prepared by reacting ethyl levulinate with 2-methyl-1,3-propanediol or a ketal or acetal of 2-methyl-1,3-propanediol to form a ketal. When ethyl levulinate is reacted with 2-methyl-1,3-propanediol, the resulting ketal product has the structure:

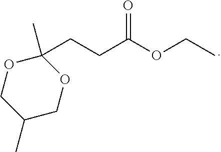

The resulting ketal can then be reacted with a polyol comprising a structure corresponding to R⁶(OH)t in a transesterification reaction. The reaction product is a compound having a structure corresponding to structure I, wherein R⁶ is as defined above. In a specific embodiment, the polyol is 2-methyl-1,3-propanediol.

Certain compounds according to structure I may exist as optical and/or geometrical isomers. In such cases, any of the isomers are suitable.

The transesterification reactions that are used to form the compounds of structure I can be carried out in the presence of an inert solvent, such as hexane, toluene, dichlorobenzene and the like. In other embodiments the reaction is carried out neat. In some embodiments, the reaction is performed at temperature and pressure conditions such that the condensation coproduct, i.e., an alcohol in most cases but water in some cases, evaporates from the reaction mixture, wherein the vapor is condensed and thereby removed. In some embodiments, a temperature between about 60° C. and 300° C. is employed; in other embodiments, a temperature of about 100° C. to 250° C. is employed; in still other embodiments, a temperature of about 160° C. to 240° C. is employed to accomplish the reaction. In some embodiments, pressure in the reaction vessel is lowered to below atmospheric pressure to assist in the removal of the condensation by-product, i.e., the alcohol or water. In some embodiments, nitrogen is sparged or swept through the reaction mixture to assist in the removal of the coproduct alcohol.

The various reactions described above are typically performed in the presence of a catalyst. While the choice of catalyst employed in the reactions is not particularly limited within the scope of the disclosure, a preferred set of embodiments employs metallic catalysts, for example, a catalyst based on titanium, aluminum, zirconium, or tin, such as titanium tetraisopropoxide (Ti(OiPr)4), or tin (II) octanoate, or organic zirconates. Other suitable catalysts are, for example, organic titanates and zirconates marketed under Tyzor® brand by DuPont deNemours and Co. of Wilmington, Del. In some embodiments, more than one species of catalyst is used; thus, blends of one or more catalysts such as those mentioned above may be used in a mixture to catalyze the formation of compounds of structure I.

In some embodiments, catalysts such as inorganic bases, including sodium methoxide, sodium ethoxide, calcium acetate, and potassium methoxide, can be used. Organo-ammonium and organo-phosphonium catalysts can be used, such as tetramethylammonium hydroxide, tetrabutyl phosphonium hydroxides and acetates Strong acid catalysts, including camphorsulfonic acid or sulfuric acid can be used in ketalization and esterification reactions. Catalysts are used in amounts suitable to catalyze the reaction. In embodiments, the amount of organometallic catalyst employed is about 5 to 50,000 ppm based on the weight of the total weight of reagents, or about 10 to 500 ppm based on the total weight of reagents.

In some embodiments, the catalyst is incorporated into, or onto, or covalently bound to, a solid support material. Resin beads, membranes, porous carbon particles, zeolite materials, and other solid support materials may be functionalized with catalytic moieties that are, in embodiments, covalently bound or strongly sorbed to one or more surfaces of the solid support.

In some embodiments, it is desirable to deactivate the catalyst after the reaction is complete. Deactivation is useful in embodiments, for example, where distillation or a high temperature process or application is to be employed. Deactivation is accomplished by any convenient technique; the method is not particularly limited by the method of deactivation. Examples of deactivating materials include compounds such as IRGAFOS® 168 and PEP-Q®, or IRGANOX® MD1024 (BASF®; Ludwigshafen am Rhein, Germany), and carboxylic acids such as salicylic acid and the like.

The various synthesis reactions described herein can be carried out batch wise or in continuous mode, depending on equipment, scale, and other reaction parameters. The reaction vessel may be made of any suitable material. In some embodiments, the reagents are dried before addition of catalyst, using any convenient technique. In embodiments, drying is accomplished by warming the reaction vessel to about 60° C.-110° C. and applying a vacuum of 5-20 Torr for at least about an hour; in other embodiments a dry inert gas, such as nitrogen or argon, is swept continuously through the vessel instead of applying a vacuum. The reagents are, in some embodiments, analyzed for water content prior to addition of catalyst to the vessel. In other embodiments, the reagents are dried separately prior to addition to the reaction vessel and are introduced to the vessel by a closed system, such as by pipes or tubes, which does not entrain water or air during introduction of the reagents to the vessel.

The catalyst may be added batchwise or in continuous fashion to the vessel. In embodiments, during the addition of catalyst, the reagents are at the same temperature as employed during drying. In other embodiments the reagents are preheated to a targeted temperature, for example in the ranges specified above, prior to addition of the catalyst. After catalyst addition, in some embodiments, a vacuum is employed to remove any air that has become entrained during the addition. In other embodiments, the catalyst is introduced to the vessel by a closed system, such as by pipes or tubes that do not entrain water or air during introduction of the reagents to the vessel. The reaction is, in embodiments, carried out under an inert gas blanket or an inert gas sparge, and agitated using any convenient means of agitation.

In embodiments, the reaction is complete in less than about 2 hours; in other embodiments the reaction is complete between about 1 hour and 12 hours; in still other embodiments the reaction is complete in about 2 to 8 hours. In some embodiments, the limiting reagent in the reaction is metered in gradually by employing an addition funnel, metered pump, or another apparatus known in the industry. Metering of a reagent is, in embodiments, initiated after or during addition of the catalyst and is particularly useful where the reaction is accomplished in a continuous process.

If desired, the crude reaction product can be purified using any convenient techniques, one of which is distillation. A distillation may be performed under reduced pressure or with the aid of nitrogen sparging. It is preferred to perform the distillation in a way that minimizes heat history. Therefore, this step is preferably performed below temperatures at which degradation, color formation, or another side reaction occurs, or if such temperatures are used, the distillation should be performed to minimize the exposure time of the product to such temperatures. In some embodiments, it is desirable to maintain temperatures at or below 200° C. during purification. In other embodiments, it is desirable to maintain temperatures at or below 175° C. during purification. Techniques such as wiped film evaporation, falling film evaporation, and membrane pervaporization are useful. Purification is carried out either with or without prior deactivation of the catalyst.

In some cases, in which mixtures of reaction products are obtained, it may be desirable to separate one or more of those reaction products from the mixture of reaction products, in order to obtain a product that is enriched in (or even consists of) a specific compound or mixture of compound. This can be performed by any suitable technique, including distillation, solvent extraction, chromatographic methods, and the like.

Compounds according to structure I are useful components in compositions that also contain an organic polymer. A very wide range of organic polymers is useful, depending of course on particular applications. The organic polymer may be thermoset or thermoplastic.

Many compounds according to structure I have Hildebrand Solubility Parameters (“HSP”) of at least 12 (MPa)½, quite typically from 12 to 20 (MPa)½. Such compounds tend to be quite compatible with organic polymers having Hildebrand Solubility Parameters (“HSP”) of about 16 (MPa)½ or greater, therefore preferred compositions are those in which the organic polymer has a Hildebrand Solubility Parameters (“HSP”) of about 16 (MPa)½ or greater. The good compatibility of these tends to make the compounds of structure I difficult to extract from the composition, and also makes the composition less likely to exude or leach the plasticizer material. Extractability in various extractants such as hexanes, soapy water, and mineral oil can be evaluated according to the ASTM D 1239 procedure; weight losses on this test are preferably no greater than 3%, no greater than 2% and still more preferably no greater than 1% for preferred compositions of the invention. Migration of a plasticizer from an article causes increased exposure of the plasticizer to the environment. The increased exposure can cause adhesive failure, cracking of materials in contact with the article, and contamination of fluids in contact with the article. Additionally, migration out of the articles can lead to stiffening, loss of performance and increase in Tg.

Some examples of suitable organic polymers include poly(vinyl chloride), poly(vinylidene chloride), polyhydroxyalkanoates, poly(lactic acid), polystyrene, polycarbonates, polyurethanes or ureas, acrylic polymers, styrene-acrylic polymer, vinyl-acrylic polymers, ethylene-vinyl acetate polymers, polyesters, polyamides, polyethers, acrylonitrile-butadiene-styrene polymers, styrene-butadiene-styrene polymers, polyvinyl acetate, poly(vinyl butyrate), polyketal esters and copolymer thereof, cellulosics, thermoplastic elastomers, or random, graft, or block copolymers thereof or mixtures thereof.

Compounds according to structure I are generally based on one or more renewable bio-based feedstocks. As such, these compounds offer opportunities to replace petroleum-based products such as plasticizer with bio-based materials. Such a bio-based compound can be blended with a bio-based organic polymer to form a polymer composition which is also bio-based. One such polymer is poly(lactic acid), or PLA. Compounds according to structure I are good plasticizers for PLA. Compound I often has high permanence in PLA compared to other compatible plasticizers.

The compounds according to structure I may be incorporated into an organic polymer composition using any suitable technique such as mechanical blending or compounding, melt blending, solution blending and the like. When the organic polymer is a thermoset, the compound may be blended into one or more precursor materials, which are subsequently cured or otherwise polymerized to form the thermosetting polymer.

A composition containing a compound according to structure I and an organic polymer may take the form of a homogeneous blend, such as a dry blend, a dispersion of one component into the other, or, in some cases, that of a continuous liquid phase into which the organic polymer is dispersed in the form of polymer particles. The mixture of the compound according to structure I and the organic polymer may form the disperse phase in an emulsion or dispersion in another material, which serves as a continuous liquid phase, as is the case with a latex.

The relative amounts of the compound of structure I and the organic polymer may vary considerably. In various embodiments, the organic polymer may constitute from 10 to 99.9%, from 30 to 96%, from 65 to 90% or from 40 to 60% of the combined weight of polymer and compound of structure I.

Compounds according to structures I often perform a plasticizing function when blended with organic polymers. When a compound of structure I is to perform such a function, it is preferably liquid at room temperature or, if a solid at room temperature, it has a glass transition temperature and/or softening temperature below room temperature, often 0° or −20° C. Plasticization is indicated by a reduction in Tg of the composition, compared to that of the neat organic polymer, and or a softening or flexibilizing effect, as indicated by a reduction in Shore hardness and/or a lowered flexural modulus, respectively. Typically, the combination of the organic polymer and the compound of structure I will have a Tg of at least 5° C. lower at least 15° C. lower, at least 30° C. lower, or at least 50° C. lower than a Tg of the neat polymer, as measured by DSC according to ASTM D3418 or other DSC method. A useful general procedure is as follows: The sample is evaluated on a TA Q200 instrument with refrigerated cooling and TA Thermal Advantage software (TA Instruments; New Castle, Del.), or equivalent, using a ramp rate of 20° C./min Samples are ramped from room temperature to 210° C. followed by a rapid quench. Samples are then reheated to 210° C. at a rate of 20° C./min Glass transition temperature is measured on the second scan.

When used to perform a plasticizing function, a compound of structure I preferably has viscosities less than about 500 centipoise (cP) at 25° C. The viscosity may be from about 1 cP to 250 cP; or about 50 cP to 200 cP at 25° C. Low viscosity provides ease of compounding into one or more polymer compositions without, for example, preheating or addition of diluents or solvents to lower viscosity and enables the creation of pastes such as plastisols.

In certain embodiments, at least a portion of compound I is in a liquid phase of the plastisol. As used herein, the term “plastisol” means a flowable suspension of polymer particles in a plasticized emulsion that forms a solid, flexible, plasticized polymer product with the addition of heat. A preferred polymer phase is polyvinylchloride) although other polymer particles can be used. A plastisol in accordance of the invention may contain from 10 to 90% by weight of a compound of structure I. Polymer plastisols are, in embodiments, poured into a mold or onto a surface where the subsequent addition of heat causes the suspension to form a solid, flexible mass. In such embodiments, it is important for the plasticizer to cause “fusing”, which means for the purposes of discussion that the polymer particle boundaries of the plastisol are broken by the effect of the plasticizer, causing mixing of the polymer on a molecular scale, wherein the effect persists to the solid state. Compounds according to structure I are often function well as “fast fusing plasticizers,” which means that they shorten the time required for the polymer particle boundaries of the plastisol to be broken and mixing to occur, lower the temperature required for the polymer particle boundaries of the plastisol to be broken and mixing to occur, or both.

Plastisols in accordance with the invention are useful in the production of sheet stock or films, flooring, tents, tarpaulins, coated fabrics such as automobile upholstery, in car underbody coatings, in moldings and other consumer products. Plastisols are also used in medical uses such as blood bags and multilayered sheets and films, tubing, footwear, fabric coating, toys, flooring products and wallpaper. Plastisols typically contain 40 to 200 parts by weight, more typically 50 to 150 parts by weight, more typically 70 to 120 parts by weight, more typically 90 to 110 parts by weight of plasticizer per 100 parts of dispersed polymer particles. PVC plastisols are usually made from PVC that has been produced by emulsion polymerization.

In certain embodiments, compounds according to structure I are contained in a PVC plastisol composition containing from 40 to 200 parts by weight, or 50 to 150 parts by weight, or 70 to 120 parts by weight, or 90 to 110 parts by weight of the compound per 100 parts of PVC. Such plastisol compositions tend to have stable viscosities; their viscosities tend to increase less than about 200% over a period of 14 days when stored at a temperature between about 20° C. to 25° C., or less than about 100%, preferably less than 70% and more preferably less than 50% when stored at a temperature of between about 20° C. to 25° C. for five days.

In another embodiment of the present disclosure, a process for the production of flexible PVC articles is provided, whereby a layer is formed from a plastisol containing from 40 to 200 parts by weight, or 50 to 150 parts by weight, or 70 to 120 parts by weight, or 90 to 110 parts by weight of a plasticizer composition containing one or more of compound I per 100 parts by weight of PVC, and subsequently fusing the layer by the application of heat.

A plastisol in accordance with the invention may further contain one or more additional plasticizers such as diethylene glycol dibenzoate, butyl benzyl phthalate, dibutyl phthalate, diisononyl phthalate, diiodecyl phthalate, other dialkyl phthalates, dipropylene glycol dibenzoate, such as the phenyl cresyl esters of pentadecyl sulfonic aromatic sulfonic acid esters available from Bayer AG of Leverkusen, Germany as MESAMOLL™, citrates such as tributylacetyl citrate, tri-2-ethylhexyl phosphate, trioctyl phosphate such as 2-ethylhexyl-isodecyl phosphate, di-2-ethylhexyl phenyl phosphate, triphenyl phosphate and tricresyl phosphate and the like.

In general, polymer compositions in accordance with the invention may further include one or more crosslinkers, adjuvants, colorants, antifouling agents, tougheners, solvents, fillers, metal particulates, odor scavenging agents, lubricants, thermal stabilizers, light stabilizers including UV stabilizers, flame retardant additives, pigments, blowing agents, processing aids, impact modifiers, coalescing solvents, or a combination thereof, including those materials described in U.S. patent application Ser. No. 13/648,252.

The useful, optional additives include, but are not limited to, trimethyl pentanyl diisobutyrate, dialkyl isophthalates, dialkyl terephthalates, alkyl benzyl phthalates, dialkyl adipates, trialkyl trimellitates, alkylyl trialkyl citrates, dialkyl azelates, dialkyl glutarates, dialkyl sebacates, dialkyl cyclohexanedicarboxylates, dialkyl sulfonates, esters of pentaerythritol, esters of glycerol, esters of fatty acids, glycol dibenzoates, epoxidized soybean oil, any of the additives described in International Patent Application Nos. PCT/US08/79337, PCT/US09/40841, or U.S. patent application Ser. No. 13/648,252 or a mixture of any of these additional additives. One or more of the alkyl, dialkyl, or trialkyl groups are, in embodiments, n-butyl, n-pentyl, n-hexyl, n-heptyl, n-octyl, n-nonyl, n-decyl, n-undecyl, n-dodecyl, capryl, cyclohexyl, 2-ethylhexyl, isobutyl, isopentyl, isohexyl, isoheptyl, isooctyl, isononyl, isodecyl, isoundecyl, or a mixture thereof. In embodiments, the alkylyl is acetyl or n-butyryl. In embodiments, the glycol is ethylene glycol, propylene glycol, diethylene glycol, or dipropylene glycol. The additional additives are present, in embodiments, as a blend with compound I.

Still more, optional materials that may be present in a polymer composition of the invention include, for example, one or more solvents (including coalescing solvents), crosslinkers, colorants (dyes or pigments), antifouling agents (such as antifungal, antibacterial, or antiviral agents), tougheners, tackifiers, additional polymers, fillers, diluents, viscosity modifying agents, metal particulates, odor scavenging agents, adjuvants, lubricants, heat stabilizers, light stabilizers including UV stabilizers, flame retardant additives, blowing agents, processing aids, impact modifiers, or a combination thereof. The additional materials impart various elements of functionality to the composition, the nature of which depend on the intended use of the composition, for example in one or more articles as will be described below.

Polymer compositions of the invention are useful to form a variety of articles. An “article” as used herein is an item with a discrete shape, such as a tube, a film, a sheet, or a fiber, that incorporates one or more compositions of the disclosure; in some embodiments, the article may have its origin in a composition that undergoes a transformation, such as solidification or evaporation of one or more solvents, to result in the final article. In some embodiments, an article is substantially formed from a polymer composition of the invention; in other embodiments, the polymer composition of the invention forms only one part, such as one layer, of an article.

An article can be formed from a polymer composition of the invention by a wide range of fabrication methods, including for example, coating, casting, extrusion, coextrusion, profile extrusion, blow molding, thermoforming, injection molding, coinjection molding, reaction injection molding, milling, or weaving. Where the polymer includes PVC, for example, the article is, in some embodiments, a casing, a pipe, a cable, a wire sheathing, a fiber, a woven fabric, a nonwoven fabric, a film, a window profile, a floor covering, a wall base, an automotive item, a medical item, a toy, a packaging container, a screw closure or stopper adapted for a bottle, a gasket, a sealing compound, a film, a synthetic leather item, an adhesive tape backing, or an item of clothing. In some embodiments, the casing is a casing for an electrical device. In some embodiments, the medical item is medical tubing or a medical bag. In some embodiments, the film is a roofing film, a composite film, a film for laminated safety glass, or a packaging film. In some embodiments, the packaging container is a food or drink container. In some embodiments, the sealing compound is for sealed glazing. In some embodiments, the automotive item is seat upholstery, an instrument panel, an arm rest, a head support, a gear shift dust cover, a seat spline, a sound-deadening panel, a window seal, a landau top, a sealant, a truck tarpaulin, a door panel, a cover for a console and glove compartment, a trim laminating film, a floor mat, a wire insulation, a side body molding, an underbody coating, a grommet, or a gasket.

In some embodiments, the article comprises two or more layers and the compound of structure I constitutes or is contained within at least one layer. In another embodiment, the article comprises a composition containing compound I in at least one layer. In some such embodiments, the other of the two adjacent layers contains a plasticizer that doesn't have a structure corresponding to compound I; the plasticizers include, in various embodiments, other additives. Some examples of such additives include dialkyl phthalates, trimethyl pentanyl diisobutyrate, dialkyl isophthalates, dialkyl terephthalates, alkyl benzyl phthalates, dialkyl adipates, trialkyl trimellitates, alkylyl trialkyl citrates, dialkyl azelates, dialkyl glutarates, dialkyl sebacates, dialkyl cyclohexanedicarboxylates, esters of pentaerythritol, esters of glycerol, fatty acid triglycerides, esters of fatty acids, glycol dibenzoates, epoxidized soybean oil, and mixtures thereof.

Certain polymer compositions in accordance with the invention are useful as adhesives, including as adhesive films or adhesive coatings. Such adhesives may include, for example, a poly(vinyl acetate) or vinyl acetate copolymer emulsion.

In some embodiments, compound I is useful as a plasticizer in nail polish formulations. In another embodiment, compound I can be used as solvents and/or cosolvents in these formulations. Polymers useful in nail polish formulations include nitrocellulose, tosylamide-formaldehydes and the like.

The following examples further elucidate and describe the compounds of the disclosure and applications thereof without limiting the scope thereof. All parts and percentages are by weight unless otherwise indicated.

### EXAMPLE 1

To a 2 liter 4-necked round bottom flask equipped with a dean-stark trap was added 250 g 2-methyl-1,3-propanediol (MPDO), 599.91 g Ethyl Levulinate (˜1.5 eq.), and 0.5 mL of Camphorsulfonic Acid solution (40% in water). The flask was heated to 100 C and placed under vacuum, starting at 85 torr and gradually lowering to 20 torr over the course of the reaction in order to maintain a steady reflux. The lower aqueous phase in the dean-stark trap was periodically drained. After 4 hours water collection had ceased, and the reaction was quenched by addition of 10.00 g Na₂HPO₄ and stirring for 18 hours while the reaction cooled. After filtration to remove the quenching agent, the reaction product was purified by fractional distillation to obtain 219.58 g of MPDO ketal of ethyl levulinate (product) (37.7% of theoretical), 99.44 purity (area % by GC-FID) and containing MPDO (0.49 area % by GC-FID). The product had the structure:

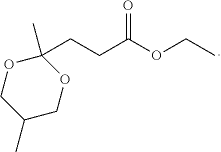

To a 500 mL 3-necked round bottom flask equipped with a dean stark trap was added 30.09 g 2-methyl-1,3-propanediol and 215.65 g of the MPDO ketal of ethyl levulinate (˜3 mole equivalents). The flask was heated to 110 C and a nitrogen sweep (0.4 scfh) was introduced into the flask to remove any residual water from the reagents. After 45 minutes the temperature was increased to 165 C and 0.05 mL of titanium tetra-isopropoxide transesterification catalyst was added, and the temperature further increased to 210 C. Distillate was periodically removed from the dean-stark trap over the course of the reaction. After 4 hours the flask was allowed to cool and stabilizers (0.28 g irganox 1010 and 0.28 g irgafos 168) were added. The product was purified by removal of low molecular weight impurities using a wiped film evaporator to obtain 139.36 g product having structure (Ia) (96.9% of theoretical) having purity of 96.46% (area % by GC-FID).

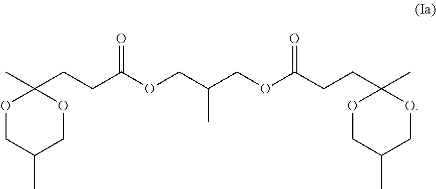 [SMILES: CC(COC(=O)CCC1(C)OCC(C)CO1)COC(=O)CCC1(C)OCC(C)CO1]

### EXAMPLE 2

Materials—HEXAMOLL DINCH (1,2-Cyclohexane dicarboxylic acid, diisononyl ester—“DINCH”) was obtained from BASF Corporation. Dioctyl phthalate (“DOTP”) was obtained from Aldrich. BBP (butyl benzyl phthalate—“BBP”) was obtained from Ferro, under the trade name Santicizer 160. Diisononyl phthalate (“DINP”) was obtained from Exxon Mobil under the trade name Jayflex. GEON 121A PVC resin is a high molecular weight micro-suspension grade (K=74) from PolyOne. Therm-Check 175 was obtained from Ferro Corporation, Cleveland, Ohio.

Liquid blends of plasticizers were prepared at the appropriate ratio (75:25) in advance or neat (100%) as shown in Table 1, and then slowly added to pre-weighed PVC resin with continuous mixing. Once resin and plasticizer were well mixed, the liquid stabilizer was added. All formulations contained 100 parts GEON 121A PVC resin, 60 phr plasticizer (single component or pre-blended), 2 phr Therm-Check 175. After mixing, formulations were degassed in a vacuum oven overnight at without heat.

Gel temperature was measured on a metal plate with a temperature gradient. Coatings (0.006 inches in width) were drawn down on the metal plate using a 6 mil drawdown bar and a piece of smooth aluminum foil was placed on top of the liquid coating. After 1 minute, the aluminum film was removed and the film was visually assessed. The gel temperature is the point at which the film transitioned from a crackled, wet film to a smooth dry film. Reported values are median temperatures measured across the width of the films.

Volatility was measured when the plastisol was poured into a metal dish and cured for 35 minutes at 180° C., resulting in films with a nominal thickness of 50 mils. The weight of the empty pan, the filled pan before cure, and the filled pan after cure were measured. The mass loss was assumed to be due to volatilization of the plasticizer and is reported in terms of % of plasticizer, which is calculated based on knowing the amount of plasticizer in the formulation, the amount of formulation added to the pan, and the amount of mass loss during the cure step.

Examples below in Table 1 show that the compound having structure (Ia) used as a plasticizer reduces the gel temperature when blended with other plasticizers. The gel temperature of DINCH, for instance, decreased from 112° C. (example 53) to 71.9° C. (example 50). The gel temperature is comparable to the competitive plasticizer, DOTP (example 52), but the compound having structure (Ia) has the advantage of being a bio-based plasticizer. Moreover, the volatility of the 75:25 blends with DINCH and the compound having structure (Ia) (example 50) show lower volatility than the comparative DINCH (example 53). Volatilization during cure can have undesirable effects, like poor film quality, odors during processing. The same trends are observed with DOTP and to a lesser extent with DINP. The comparative BBP also works for reducing gel temperature of DINP (example 51) but it has several disadvantages that make it an unattractive option: it is a phthalate, it is listed as a danger to human health on California's Proposition 65 list, and it is being banned in Europe, effective 2015.

TABLE 1

| | | Blend ratio | | | Shore | | % |
|---|---|---|---|---|---|---|---|
| Ex- | | (plasticizer | Com- | Com- | A | Gel T | vola- |
| am- | | 1: plas- | ponent | ponent | Hard- | | tility, |
| ple | PHR | ticizer 2) | 1 | 2 | ness | (° C.) | % of pz |
| 2 | 70 | 100:0 | Com- | — | 68.2 | 68.1 | 3.92% |
| | | | pound | | | | |
| | | | (Ia) | | | | |
| 3 | 70 | 100:0 | DINP | — | 71.0 | 101.4 | 4.42% |
| 4 | 70 | 100:0 | DOTP | — | 69.0 | 109.0 | 6.99% |
| 5 | 70 | 100:0 | DINCH | — | 68.1 | 120.4 | 8.13% |
| 6 | 60 | 75:25 | DOTP | Com- | 72.2 | 70.8 | 6.92% |
| | | | | pound | | | |
| | | | | (Ia) | | | |
| 7 | 60 | 75:25 | DINCH | Com- | 74.0 | 71.9 | 7.08% |
| | | | | pound | | | |
| | | | | (Ia) | | | |
| 8 | 60 | 75:25 | DINP | BBP | 74.2 | 68.4 | 7.68% |
| 9 | 60 | 100:0 | DOTP | — | 76.4 | 73.5 | 6.94% |
| 10 | 60 | 100:0 | DINCH | — | 75.1 | 112.4 | 8.66% |

### EXAMPLES 11-12

Extraction of Plasticizer with Water from Blends of Compounds

The plasticizer extraction from films prepared from plastisols was done according to ASTM D 1239-98. Films were prepared by drawing down plastisols having 70PHR of plasticizer and 2 PHR of Therm-Check 175 on an aluminum foil with a 10 mils draw down bar. The films were cured for 20 min at 140° C. They were then cooled down in a desiccator to avoid water uptake in humid environment. Then a sample of 1 g was weighed out on an analytical balance with a precision of 0.1 mg. It was placed into a 250 ml glass bottle filled with 200 ml of deionized water, fully immersed into the water phase and left for 24 hours at ambient temperature. The sample was removed from the bottle and wiped with a paper towel to remove physically attached water and dried in an oven at 105° C. for 1 hour, cooled down in a desiccator and weighed again (weight C).

The plasticizer extraction by water was calculated by % weight loss and is shown below in Table 2.

TABLE 2

| | | | % weight loss, DI |
|---|---|---|---|
| Example | PHR | Component | extraction |
| 11 | 70 | Compound (Ia) | 1.43% |
| 12 | 70 | DINP | 0.23% |

This shows a surprisingly low water extraction property for Compound (Ia), which makes it a desirable blend component for systems where good (low) water extraction properties are desirable.

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

1. A compound having a structure corresponding to structure I 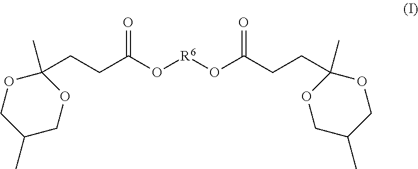
wherein R⁶ is a hydrocarbyl group or a substituted hydrocarbyl group.

2. The compound of claim 1, wherein R⁶ contains one or more ether or ester groups.

3. The compound of claim 1, wherein R⁶ is C₂-C₆ alkyl.

4. The compound of claim 1, which has the structure:

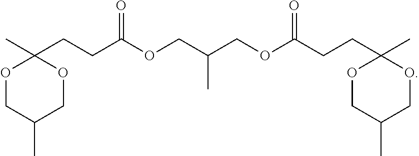 [SMILES: CC(COC(=O)CCC1(C)OCC(C)CO1)COC(=O)CCC1(C)OCC(C)CO1]

5. A mixture comprising two or more compounds of claim 1.

6. A composition comprising the compound of claim 1, and an organic polymer.

7. The composition of claim 6, which has a glass transition temperature at least 30° C. lower than a glass transition temperature of the polymer.

8. The composition of claim 6, wherein the compound constitutes from 0.1 to 90% of the combined weight of the compound and the polymer.

9. The composition of claim 6, wherein the polymer is a thermoplastic.

10. The composition of claim 6, wherein the polymer is a thermoset.

11. The composition of claim 6, wherein the polymer comprises a poly(vinyl chloride), polyhydroxyalkanoate, a poly(lactic acid), a polystyrene, a polyurethane, a polyurea, a polyurea-urethane, a polycarbonate, an acrylic polymer, a styrene-acrylic polymer, a vinyl-acrylic polymer, an ethylene-vinyl acetate polymer, a polyester, a polyamide, a polyether, a polybutadiene, an acrylonitrile-butadiene-styrene copolymer, a styrene-butadiene-styrene copolymer, a polyvinyl acetate, an elastomer, or homopolymers thereof, or random, graft, or block copolymers thereof, or blends or mixtures thereof.

12. The composition of claim 6, wherein the compound is melt blended or solution blended with the polymer.

13. The composition of claim 6, wherein the composition is a plastisol.

14. The composition of claim 6, wherein the composition is a dry blend.

15. The composition of claim 6, wherein the polymer is a poly(vinyl chloride).

16. The composition of claim 6, further comprising one or more crosslinkers, adjuvants, colorants, antifouling agents, tougheners, solvents, fillers, metal particulates, odor scavenging agents, lubricants, thermal stabilizers, light stabilizers including UV stabilizers, flame retardant additives, pigments, blowing agents, processing aids, impact modifiers, coalescing solvents, antioxidant or a combination of any two or more thereof

17. The composition of claim 6, further comprising one or more additives selected from the group consisting of dialkyl phthalates, trimethyl pentanyl diisobutyrate, dialkyl isophthalates, dialkyl terephthalates, alkyl benzyl phthalates, dialkyl adipates, trialkyl trimellitates, alkylyl trialkyl citrates, dialkyl azelates, dialkyl glutarates, dialkyl sebacates, dialkyl cyclohexanedicarboxylates, esters of pentaerythritol, esters of glycerol, fatty acid triglycerides, esters of fatty acids, glycol dibenzoates, epoxidized soybean oil, and mixtures thereof

18. The composition of claim 6, wherein the composition has a plasticizer extraction by water of less than 3% as per ASTM D 1239-98.

19. An article comprising the composition of claim 6.

20. A process for plasticizing a polymer comprising melt or solution blending a polymer and a plasticizing amount of at least one compound of claim 1.

21. A method of making a compound of claim 1, comprising:
a. reacting ethyl levulinate with 2-methyl-1,3-propanediol or a ketal or acetal of 2-methyl-1,3 -propanediol; and
b. adding a polyol comprising a structure corresponding to R⁶(OH) under reaction conditions to the product of step (a) to provide a compound having a structure corresponding to claim 1, wherein R⁶ is as defined in claim 1.

22. The method of claim 21, wherein ethyl levulinate is reacted with 2-methyl-1,3-propanediol.

23. The method of claim 21, wherein ethyl levulinate is reacted with a ketal or acetal of 2-methyl-1,3-propanediol.

24. The method of claim 21, wherein the polyol is 2-methyl-1,3-propanediol.

25. A compound having the structure [chemical structure]
